(19) United States
(12) Patent Application Publication
(10) Pub. No.: US 2004/0013710 A1
(43) Pub. Date: Jan. 22, 2004

(54) Hydrolyzable tannin extracts from plants effective at inhibiting bacterial adherence to surfaces

(76) Inventor: Amy B. Howell, Hammonton, NJ (US)

Correspondence Address: LICATLA & TYRRELL P.C., 66 E. MAIN STREET, MARLTON, NJ, 08053, US

(21) Appl. No.: 10/332,425

(22) PCT Filed: Jul. 20, 2001
(86) PCT No.: PCT/US01/22973

(51) Int. Cl. A61K 31/7024; A23K 1/165; A23K 1/17

(52) U.S. Cl. 424/442; 514/23; 536/110

(57) ABSTRACT

Hydrolyzable tannin extracts from plants are provided which have activity to prevent bacterial adherence to surfaces. Methods for prevention and treatment of urinary tract and kidney infections in animals and humans are provided.

### BACKGROUND OF THE INVENTION

Millions of women each year are diagnosed with cystitis (bladder infections) and pyelonephritis (kidney infection). Countless numbers of companion animals also suffer from chronic urinary infections and die from renal infection. E. coli bacteria is the most common pathogen associated with these infections, causing over 80% of urinary tract infections. Over 30% of women suffer recurrent infections within a 6 to 12 month period and are forced to resort to extended use of antibiotics to treat these infections. Recurrent use of antibiotics can lead to pathogen resistance and result in deleterious side effects and toxicity. Consequently, there exists a need for safe alternative medications (e.g., non-antibiotics) that can be used to prevent or treat urinary tract infections in both animals and humans.

In order to initiate a urinary tract infection, bacteria must attach to epithelial cells so that they can multiply and induce symptoms of disease. In the mammalian body, fluids such as tears, saliva and urine would transport bacteria out of the body if adherence to cell surfaces did not occur. In the case of E. coli attachment to the epithelial cells that line the kidney and bladder, bacterial adherence is facilitated by fimbriae, which are proteinaceous fibers on the bacterial cell wall. These fimbriae produce adhesins that attach to specific monosaccharide or oligosaccharide receptor sequences on the urinary epithelial cells. The bacteria reach the cell surface and bind to these receptors. Once attached, the bacteria are able to multiply and deliver their toxins to susceptible host cells resulting in tissue damage that is associated with urinary tract infections.

E. coli isolates can differ in the type of fimbrial adhesin that they produce, allowing them to attach to cell surface receptors which are specific for the particular adhesin. These fimbriae have been identified and characterized on the basis of their molecular structure, antigenic type, and receptor specificity. Two fimbrial types (type 1 and P-type) are morphologically identical, but they differ antigenically and in their attachment sites on cell surfaces. Type 1 E. coli have fimbriae that bind to a receptor that is the carbohydrate (mannose-containing) part of a glycoprotein found on epithelial cell surfaces in the urinary tract. P-type E. coli fimbriae attach to a carbohydrate receptor (a disaccharide with a Gal-Gal sequence) on glycosphingolipids located on the surface of urinary epithelial cells.

Cranberry juice has been shown to reduce bacteriuria associated with urinary tract infections in humans, an effect that appears to be due to the ability of certain cranberry compounds to inhibit bacterial adhesion of both type 1 and P-type E. coli bacterial phenotypes to human bladder epithelial, or uroepithelial cells (Sobota. 1984. J. Urol. 131:1013-1016; Schmidt and Sobota. 1988. Microbios. 55:173-181; Zafriri. 1989. Antimicrob. Agents Chemother. 33:92-98; Howell, A. B. et al. 1998. New Engl. J. Med. 339:1085-1086). Type I adherence is inhibited by fructose found in many juices (Zafriri. 1989. Antimicrob. Agents Chemother. 33:92-98). A partially purified anti-adherence extract from cranberry has also been described (U.S. Pat. Nos. 5,474,774; 5,525,341; and 5,646,178), which with further purification was reported to be a fraction enriched in polyphenol and flavonoid compounds that contained as much as 10% anthocyanins. A series of singly-linked B-type proanthocyanidin monomers, dimers, polymers, and flavonoid derivatives thereof have been reported to have the ability to interfere with bacterial adherence to surfaces (WO 96/30033; U.S. Pat. Nos. 5,646,178 and 5,650,432). However, no specific compound was identified as responsible for the biological activity, nor was the specificity of this adhesion activity for P-type or type 1 bacteria determined. It has been demonstrated that condensed tannins (proanthocyanidins) with A-type double linkages isolated from cranberries have activity to inhibit adherence of P-type E. coli to uroepithelial cells, but not to type I E. coli (Foo, L. Y. et al. 2000. Phytochemistry 54:173-181).

The role of pomegranate extracts as anti-microbial compounds has also been explored (Chulasiri, M. et al. 1995. Mahidol. Iniv. J. Pharm. Sci. 22:1-159; Chulasiri, M. 1997. Thai. J. Phytopharm. 4:25-30; Stewart, G. S. et al. 1998. J. Appl. Microbiol. 84:777-783; Segura, J. J. et al. 1990. Arch. Invest. Med. 21:235-239). However, in each case, the pomegranate extract was shown only to inhibit bacterial growth, not to inhibit bacterial adherence to surfaces.

It has now been found that hydrolyzable tannin extracts from plants, in particular pomegranate and persimmon, have activity to inhibit adhesion of bacteria to surfaces, a biological effect that makes these compounds useful for prevention and/or treatment of urinary tract infections and kidney infections in animals and humans, as well as to prevent or reduce bacterial adherence to cellular surfaces that are associated with infections.

### SUMMARY OF THE INVENTION

Compositions comprising hydrolyzable tannin extracts of plants which inhibit adherence of bacteria to cellular surfaces and methods for preventing or treating bacterial infections are provided.

### DETAILED DESCRIPTION OF THE INVENTION

Tannins are phenolic metabolites of relatively high molecular weight found in plants (e.g,. fruit, leaves, stems, roots, and bark). The following plants listed by their genus have been shown to contain high concentrations of hydrolyzable tannins: Punica (pomegranate), Diospyros (persimmon), Rubus (blackberry), Fragaria (strawberry), Vitis (grape), Ribes (currant), Pinus, Eucalyptus, Cacao, tea, Vaccinium, Sanguisorba, Tibouchina, Myrobolan, Geum, Casuarina, Davidia, Quercus, Agrimonia, Potentilla, Rosa, Terminalia, Rheum, Fuschia, Ceratonia, Bergenia, Caesalpinia, Acer, Rhus, Cotinus, Geranium, Hamamelis, Castanea, Euphorbia, Eugenia, Pelargonium, Paeonia, Parrottia, and Coriaria. Plant-extracted tannins are classified into two distinct groups of metabolites, the condensed tannins (proanthocyanidins) and hydrolyzable tannins. The condensed tannins consist of chains of flavan-3-ol units. The hydrolyzable tannins contain a polyhydric alcohol core, normally glucose, which is esterified with one or more gallic acid units to form the gallotannins, or with hexahydroxydiphenic acid to form the ellagitannins.

It is believed that the hydrolyzable tannins contain structures similar to the bacterial-binding receptors found on the surface of bladder and kidney cells. Therefore, these compounds act, not by killing the bacteria directly, but rather by binding bacterial fimbriae and thereby preventing adherence of the bacteria to bladder or kidney cell surface receptors. Without binding to the cells, the bacteria cannot multiply, steps apparently necessary to cause a urinary tract infection. The unbound bacteria are thus carried harmlessly out of the body in the urine stream. This type of anti-adherence mechanism is advantageous since it reduces the selective pressure to develop antibiotic resistance that can occur during multiplication of bacteria in the presence of antibiotics.

In addition to use in the prevention and treatment of urinary tract infection in animals and humans, the hydrolyzable tannins extracted from fruit would have broad use to prevent adherence of bacteria to any cellular surface. For example, the hydrolyzable tannin extract from plants that would include but not be limited to pomegranate and persimmon can be used as a food additive for cattle, and other livestock animals used for food production, to reduce E. coli in the digestive tract of the animals and to lead to a reduction in the contamination of meat. Other uses would include but not be limited to use to reduce infection after surgery, use as a treatment for topical wounds (e.g., acne), use to prevent or treat bacterial oral infections, and use to prevent or treat urinary tract infection in dogs, cats, and other mammals.

In a preferred embodiment, the hydrolyzable tannins were prepared by extracting pomegranates. A five-fold concentrate of pomegranate juice was prepared by pressing juice from the pomegranate and then concentrating the juice. To remove fats and waxes, solid phase C18 column chromatography or liquid partitioning was performed with petroleum ether. In the C18 column method, columns were conditioned with 1 column volume of methanol, followed by 1 column volume of water. The thoroughly mixed extract was then loaded onto the column. To remove simple sugars, the column was washed with 2 column volumes of water. To remove organic acids, the column was washed with 2 column volumes of 15% methanol. All wash fluids were discarded. The polyphenolic fraction was then eluted with 3 column volumes of methanol acidified with 1% acetic acid and dried under reduced pressure to remove solvents. Alternatively, a partition method can be used to remove fats and waxes where equal volumes of pomegranate juice and petroleum ether were loaded into a separatory funnel, shaken gently, and after a period of settling, the water soluble fraction was eluted and repartitioned a second time with petroleum ether and dried under reduced pressure. Then, either the solid phase C18 column eluate or the partitioned fraction were subjected to further column chromatography to separate oligomeric hydrolyzable tannins from low molecular weight pigments and flavonol glycosides.

A column was loaded with Sephadex LH-20 sorbent (hydroxypropylated gel filtration column, Amersham Pharmacia, Piscataway, N.J.). The column was then equilibrated overnight in 50% ethanol. The C18 column eluate fractions or the partitioned fractions were then suspended in 50% ethanol. After vortexing, the fractions were immediately loaded onto the LH-20 column. Extraneous low molecular weight material was first removed by washing the column with 10 column volumes of 50% ethanol or with sufficient column volume to remove all red color. Rinses were discarded. Hydrolyzable tannins were then eluted with 8 column volumes of 70% acetone:30% water. The fractions were evaporated under reduced pressure to remove all solvent. The resulting light brown tannin powder was freeze-dried and stored in an airtight container at 4° C. in the dark to minimize oxidation. ¹³C-nuclear magnetic resonance (NMR) imaging was used to insure that the freeze-dried fraction was pure, containing only hydrolyzable tannins. The NMR spectrum revealed that the pomegranate concentrate consisted of only hydrolyzable tannins, with mixtures of gallo- and ellagi-tannins present. There were no significant NMR signals that could be attributed to proanthocyanidin structures. Thin layer chromatography was also performed on the pomegranate concentrate fraction and only hydrolyzable tannins were identified.

The hydrolyzable tannin fraction was tested in vitro for bacterial anti-adherence properties using well established bioassay methods (DeMan, P. Et al. 1987. J. Clin. Microbiol. 25:401-406; Evans, D. G. et al. 1977. Infect. Immun. 18:330-337; Sobota, A. E. 1984. J. Urol. 131:1013-1016). The fraction was tested for the ability to: a) prevent bacterial adherence to uroepithelial cells; b) prevent bacterial-induced hemagglutination of human A(+) or O(+) erythrocytes; and c) prevent bacterial-induced agglutination of P-receptor-coated resin beads. In all bioassays, the hydrolyzable tannin extract from pomegranates and persimmons was capable of inhibiting bacterial adherence to surfaces. These data provide evidence that plants which contain high levels of hydrolyzable tannins would be useful for inhibiting bacterial adherence to surfaces.

In one embodiment, the invention is a method of preventing or treating infections, in particular urogenital infections, in an animal or human by administering a composition comprising the hydrolyzable tannin extract from plants (e.g., pomegranate or persimmon), including pharmaceutically acceptable salts of any of the hydrolyzable tannin compounds or polymers with a pharmaceutically acceptable carrier, to the animal or human in an amount and for a time sufficient to prevent, reduce, or eliminate the symptoms associated with such infections, thereby ameliorating or eliminating the infection. The composition can be administered in the form of a pharmaceutical composition or a food product or supplement. When a pharmaceutical composition is used, the invention is directed to a method of treating infections, in particular urogenital infections. When a food composition is employed, the invention is directed to a method of preventing or treating infections, in particular urogenital infections.

Using the method of the present invention, one of skill would understand how to formulate and administer the compositions based on knowledge of one of skill that is routine in the art. Treatment with the compositions of the instant invention will render bacteria non-pathogenic and unable to colonize in the urinary tract. Therefore, one measure of efficacy will be to monitor reduction or elimination of urinary bacterial counts associated with such infections during or after the course of treatment.

The compositions can be provided together with a pharmaceutically acceptable carrier. The compositions can be provided in tablet or liquid form, as an oral rinse, as a douche, as a topical formulation, as a toothpaste, or as an additive for a beverage or other food item.

The food compositions of the invention contain the hydrolyzable tannin extract of the invention in admixture with livestock feed, domestic animal feed or with a consumable food product for human consumption. Those food compositions which contain livestock feed are for cattle, pigs, and the like. Those food compositions which contain domestic animal feed are for dogs, cats, horses, and the like. Those food compositions that contain a consumable food product are for humans. The food compositions, especially beverages, can be used as therapeutics to prevent or treat urogenital infections. Alternatively, the food compositions can be general consumables, for example, ground meat or other meat product, beverages, especially juice beverages, whether or not pasteurized, grain products, fruit products, and the like.

The preferred dosage range would be determined based on results of pharmacological dose-response studies either in vitro or in vivo. Such extrapolation for dosage determination is routine in the art.

In another embodiment, the present invention is a method of inhibiting adherence of bacteria, such as E. coli, to a surface which comprises contacting said bacteria with a plant extract containing hydrolyzable tannins, prior to or concurrently with contact of said bacteria with a surface. The surface can be any substance or material, synthetic or biological, where it is desired to prevent bacterial contamination, accumulation, or infection. In a preferred embodiment, the surface is a cellular surface such as the uroepithelial cell surface, cells exposed in a wound, or on the skin or another surface such as teeth or a prosthetic device or implant. Also in a preferred embodiment the plant extract is an exptract of pomegranate.

The hydrolyzable tannins of the invention can also be used for reducing or treating infection after surgery, treating topical wounds or acne, or preventing or eliminating oral infection by administering a composition of the invention to the site of infection or potential infection in a patient. The composition is administered to the patient in accordance with the treatment being rendered. For example, it can be applied to a surgical incision or other opening as a liquid, topical cream, or by any other suitable delivery means. For topical wounds, the pharmaceutical composition can be a topical cream, solve or spray. Oral infection can be treated by brushing with a toothpaste or by using a oral rinse or mouth wash formulated with hydrolyzable tannins in accordance with the invention.

The following nonlimiting examples are provided to better illustrate the present invention.

### EXAMPLES

### Example 1

### Pomegranate Extraction Procedure

Whole pomegranate fruit was pressed to remove juice from the arils. The remaining fruit material following juice extraction is referred to as “the press cake”. Leaves, stems or roots were separately blended with 80% acetone and subjected to further chromatography.

Juice from the arils was diluted in water and applied directly to a reverse-phase C-18 chromatography column. The press cake was homogenized in 80% aqueous acetone, filtered, the supernatant dried under reduced pressure to remove solvent, and applied to a C-18 column. Fats and waxes were removed by binding lipids to the C-18 columns. The columns were conditioned with 1 column volume of methanol, followed by 1 column volume of water. The C-18 columns were loaded with the extracts and simple sugars were removed by washing columns with 2 column volumes of water. Organic acids were removed by washing with 2 column volumes of 15% methanol. The polyphenolic fractions were eluted with 3 column volumes of methanol, acidified with 1% HCl. To separate oligomeric proanthocyanidins and hydrolyzable tannins from the low molecular weight pigments, flavonol glycosides, etc., chromatography columns were loaded with Sephadex LH-20 sorbent. The columns were equilibrated overnight in 50% ethanol. The C-18 column fractions were suspended in 50% ethanol and loaded onto LH-20 columns. Low molecular weight materials (anthocyanins, flavonol glycosides, etc.) were removed by washing columns with 10 column volumes of 50% ethanol or until the red color was removed. Tannins were eluted with 8 column volumes of 70% acetone, 30% water and evaporated under reduced pressure to remove solvent.

### Example 2

### Assays for Detecting Bacterial Anti-Adherence Activity

All plant fractions were tested for anti-adherence bioactivity.

### Agglutination Assays that Detect Bacterial Anti-Adherence

Detection of type 1 anti-adherence requires a cell surface that harbors mannose-containing receptors, such as guinea pig red blood cells or yeast cells. When the type 1 fimbriae bind to the mannan on the cells, they cause cells to agglutinate. This agglutination can be prevented by treatment of the bacteria with mannose or fructose. For the bioassay, guinea pig red blood cells were suspended (3%) in phosphate-buffered saline. The cells were then mixed with type 1 bacterial suspension (5×10⁸ bacteria/ml phosphate buffered saline) that had been pre-incubated with the fruit fractions to be tested for anti-adherence activity. Alternatively, yeast cells (Saccaromyces cerevisiae) were suspended (4×10⁸ cells/ml phosphate buffered saline) and then mixed with type 1 bacterial suspension (5×10⁸ cells/ml phosphate buffered saline) that had been pre-incubated with a fruit fraction to be tested for anti-adherence activity.

To test for P-type bacterial anti-adherence activity, fruit fractions (200 μl of each) were evaporated under reduced pressure, dissolved in 1.7 ml of phosphate buffered saline, and neutralized with 1 N NaOH. Serial 2-fold dilutions of each fraction were prepared. A 30 μl drop of each dilution was incubated with 10 μl of bacterial suspension on a 24-well polystyrene plate for 10 minutes at room temperature on a rotary shaker. Freshly-drawn red blood cells donated by human volunteers with A+ blood type (HRBC) or latex beads coated with putative P-receptor were suspended (3%) in phosphate buffered saline (PBS) and added (10 μl drops) to each test suspension. Suspensions were incubated for 20 minutes on a rotary shaker at room temperature and then evaluated microscopically for agglutination. Controls included wells containing bacteria+PBS, HRBC+PBS, bacteria+test fractions, HRBC+test fractions, and bacteria+HRBC. If agglutination was inhibited, the fraction was considered to be bioactive and capable of preventing bacterial adherence.

### Preventing Direct Bacterial Adherence to Uroepithelial Cells

The sediment obtained from midstream, morning urine was washed with PBS and resuspended in PBS to a concentration of 10⁵ with a hemocytometer. Some cells were stained with methylene blue and checked for adhering background bacteria. Epithelial cells were pre-incubated with the fruit fractions, bacteria or urine (controls) for 30 minutes at 37 C. Samples (1 ml) were removed using a tuberculin syringe and filtered through an 8 micrometer polycarbonate membrane filter. The filter was then washed with 30 ml of distilled water, removed, and pressed on a glass slide. The filter was dried, removed and the cells remaining on the slide were stained with methylene blue. The bacteria adhering to the cells were counted microscopically and recorded. Fruit fractions which prevented the bacteria from adhering to the cells were considered bioactive.

What is claimed is:

1. A hydrolyzable tannin extract of a plant wherein said extract inhibits adherence of bacteria to cellular surfaces.

2. A composition comprising a tannin extract of claim 1 which is admixed with a food.

3. A composition comprising the extract of claim 1 and a pharmaceutically acceptable vehicle.

4. A method of treating an infection in an animal or human comprising administering the composition of claim 3 to said animal or human in an amount sufficient to prevent, reduce or eliminate symptoms associated with said infection.

5. The method of claim 4 wherein the infection is a urogenital infection.

6. A method of preventing or treating an infection in an animal comprising administering the composition of claim 2 to said animal in an amount and for a time to prevent, reduce or eliminate the symptoms associated with said infection.

7. The method of claim 6 wherein the infection is a urogenital infection.

8. A method of reducing pathogenic E. coli in the digestive tracts of cattle comprising administering the composition of claim 2 to said cattle.

9. A method of inhibiting bacterial adherence to a surface comprising contacting said surface with the extract of claim 1 so that bacterial adherence is reduced or eliminated.

10. A method of reducing or preventing infection after surgery in a patient comprising administering the composition of claim 3 to the site of infection or potential infection in said patient.

11. A method of treating infection or potential infection in areas of topical wounds in a patient comprising administering the composition of claim 3 to the site of the topical wound in said patient.

12. A method of reducing or preventing oral bacterial infection in a patient comprising administering the composition of claim 3 to the site of infection or potential infection in said patient.
